(19) United States
(12) Patent Application Publication
(10) Pub. No.: US 2026/0106558 A1
(43) Pub. Date: Apr. 16, 2026

(54) TRIBOELECTRIC NANOGENERATOR AND OPERATION METHOD THEREOF

(71) Applicant: NATIONAL TAIWAN UNIVERSITY, TAIPEI (TW)

(72) Inventor: Zong-Hong LIN, TAIPEI (TW)

(21) Appl. No.: 19/055,519

(22) Filed: Feb. 18, 2025

(30) Foreign Application Priority Data
Oct. 14, 2024 (TW) 113139037

(51) Int. Cl. H02N 1/04; B82Y 15/00; G01N 27/00; G01N 33/483

(52) U.S. Cl. CPC H02N 1/04; B82Y 15/00; G01N 27/002; G01N 33/483

(57) ABSTRACT

A triboelectric nanogenerator includes first and second stacks and an electricity meter. The first stack includes a first conductive layer and an amino acid layer. The second stack is disposed over the first stack and includes a negative friction layer and a second conductive layer disposed on the negative friction layer. The electricity meter is electrically connected to the first and second conductive layers. Another triboelectric nanogenerator includes an encapsulation container, a filler, an electrode, a wire, and an electricity meter. The encapsulation container has an accommodating space and a friction layer. The filler is contained in the accommodating space. The filler includes water, an electrolyte, or a liquid metal. The electrode penetrates through the encapsulation container and contacts the filler. The wire is electrically connected to the electrode and has a grounded end. The electricity meter is disposed on the wire.

### CROSS-REFERENCE TO RELATED APPLICATION

This application claims priority to Taiwan Application Serial Number 113139037, filed Oct. 14, 2024, which is herein incorporated by reference.

### BACKGROUND

### Field of Invention

The present disclosure relates to a triboelectric nanogenerator and an operation method thereof.

### Description of Related Art

Traditional detection devices usually need to be connected to an external power supply or equipped with a battery and have problems such as poor sensitivity, low stability, and expensive detection costs. For example, circular dichroism (CD) and optical rotatory dispersion (ORD) can be used to detect the chiral properties of molecules. However, the above techniques require expensive detection equipment and professional operating skills, which limits their applicability. In addition, connecting a detection device to an external power source or equipping it with a battery may easily cause inconvenience in using the detection device, increase cost, and limit its service life. Given the above, there is a need to provide a low-cost and simple detection device.

### SUMMARY

The present disclosure provides a triboelectric nanogenerator that includes a first stack, a second stack, and an electricity meter. The first stack includes a first conductive layer and an amino acid layer disposed on the first conductive layer. The second stack is disposed over the first stack, in which the second stack includes a negative friction layer and a second conductive layer disposed on the negative friction layer. The electricity meter is electrically connected to the first conductive layer and the second conductive layer.

In some embodiments, the negative friction layer includes polytetrafluoroethylene (PTFE), fluorinated ethylene propylene (FEP), polydimethylsiloxane (PDMS), silicone, poly(butylene adipate-co-terephthalate) (PBAT), polyvinylidene difluoride (PVDF), polyimide (PI), polystyrene (PS), polycarbonate (PC), or combinations thereof.

In some embodiments, the amino acid layer includes a L-type amino acid or a D-type amino acid.

In some embodiments, the amino acid layer includes glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline, or combinations thereof.

In some embodiments, the negative friction layer has a thickness of 10 nm to 1000 nm.

In some embodiments, the amino acid layer has a thickness of 1 nm to 1000 nm.

In some embodiments, a work function of the negative friction layer is higher than a work function of the amino acid layer.

The present disclosure provides a method of operating a triboelectric nanogenerator, and the method includes the following operations. The triboelectric nanogenerator of any one of the aforementioned embodiments is received. The amino acid layer of the first stack is brought into contact with the negative friction layer of the second stack. The first stack and the second stack are separated. The first stack and the second stack are brought closer to each other. A value on the electricity meter is read.

In some embodiments, the value is a current value, a voltage value, or both.

The present disclosure provides a triboelectric nanogenerator that includes a packaging container, a filler, an electrode, a wire, and an electricity meter. The packaging container has an accommodation space and a friction layer. The filler is contained in the accommodation space and in contact with the friction layer, in which the filler includes water, an electrolyte, or a liquid metal. The electrode penetrates the packaging container and is in contact with the filler. The wire is electrically connected to the electrode and has a grounded end. The electricity meter is disposed on the wire.

In some embodiments, the friction layer includes polytetrafluoroethylene (PTFE), fluorinated ethylene propylene (FEP), polydimethylsiloxane (PDMS), silicone, poly(butylene adipate-co-terephthalate) (PBAT), polyvinylidene difluoride (PVDF), polyimide (PI), polystyrene (PS), polycarbonate (PC), or combinations thereof.

In some embodiments, the friction layer has a plurality of protrusions protruding outwardly.

In some embodiments, the filler is a salt solution, and the salt solution includes a salt in a concentration of 0.05 wt % to 10 wt %.

In some embodiments, the filler comprises water, an electrolyte, or a liquid metal.

In some embodiments, the electrolyte comprises a salt solution, an ionic liquid, an acidic solution, or an alkaline solution.

In some embodiments, the friction layer is a top portion of the packaging container and has a substantially flat upper surface.

The present disclosure provides a method of operating a triboelectric nanogenerator, and the method includes the following operations. The triboelectric nanogenerator of any one of the aforementioned embodiments and an object to be tested are received. The friction layer of the triboelectric nanogenerator is brought into contact with the object to be tested. The triboelectric nanogenerator and the object to be tested are separated. The triboelectric nanogenerator and the object to be tested are brought closer to each other. A value on the electricity meter is read.

In some embodiments, the value is a current value, a voltage value, or both.

### BRIEF DESCRIPTION OF THE DRAWINGS

The present disclosure can be more fully understood by reading the following detailed description of the embodiments, with reference made to the accompanying drawings.

FIG. 1 is a flow diagram of a method of operating a triboelectric nanogenerator according to various embodiments of the present disclosure.

FIG. 2 is a schematic diagram of various intermediate stages of operating a triboelectric nanogenerator in accordance with various embodiments of the present disclosure.

FIG. 3 is a flow diagram of a method of operating a triboelectric nanogenerator according to various embodiments of the present disclosure.

FIG. 4 is a schematic diagram of various intermediate stages of operating a triboelectric nanogenerator in accordance with various embodiments of the present disclosure.

FIG. 5 is a perspective view of a packaging container of a triboelectric nanogenerator according to various embodiments of the present disclosure.

FIG. 6 shows transfer charge versus time plots of various amino acids.

FIG. 7 shows work functions and transfer charge densities of various amino acids.

FIG. 8 shows a transfer charge versus time relationship diagram, transfer charge densities, and an output voltage versus time relationship diagram of D-aspartic acid and L-aspartic acid.

FIG. 9 shows a V/V_{RT} versus temperature relationship diagram when sodium chloride solutions of different concentrations and deionized water are used as fillers of the triboelectric nanogenerators and shows an output voltage versus temperature relationship diagram of a triboelectric nanogenerator under different stretching degrees.

FIG. 10 shows an open circuit voltage versus time relationship diagram and a short-circuit current versus time relationship diagram of a triboelectric nanogenerator at different temperatures.

FIG. 11 shows an output voltage versus temperature relationship diagram of a triboelectric nanogenerator under different forces.

FIG. 12 shows an output voltage versus time relationship diagram and an average open circuit voltage diagram of a triboelectric nanogenerator when measuring different objects to be tested.

FIG. 13 shows a surface potential diagram of various objects to be tested at different temperatures and an output voltage versus temperature relationship diagram of different objects to be tested.

FIG. 14 shows output voltage versus time relationship diagrams of a triboelectric nanogenerator when measuring various objects to be tested.

### DETAILED DESCRIPTION

The following embodiments are disclosed with accompanying diagrams for detailed description. For illustration clarity, many details of practice are explained in the following descriptions. However, it should be understood that these details of practice do not intend to limit the present disclosure. That is, these details of practice are not necessary in parts of embodiments of the present disclosure. Furthermore, for simplifying the drawings, some of the conventional structures and elements are shown with schematic illustrations.

Although below using a series of operations or steps described in this method disclosed, the order of these operations or steps shown should not be construed to limit the present disclosure. For example, certain operations or steps may be performed in different orders and/or concurrently with other steps. Moreover, not all steps must be performed in order to achieve the depicted embodiment of the present disclosure. Furthermore, each operation or procedure described herein may contain several sub-steps or actions.

The present disclosure provides a triboelectric nanogenerator and an operation method thereof. The triboelectric nanogenerator can convert mechanical energy into electrical energy through triboelectric effect, thereby distinguishing different objects to be tested and identifying subtle temperature differences through output voltage and/or output current, and has advantages of high sensitivity, high stability, low cost, and a self-powered property. For example, the triboelectric nanogenerator can identify different types and/or different chiral amino acids. The triboelectric nanogenerator has a wide range of applications. For example, it can be used in health monitoring devices (such as wearable devices or implantable devices), can be installed on robots, mechanical fingers, or bionic prosthetics for sensing, or can be used in environmental monitoring, such as air quality testing, water quality testing, or pollutant testing. In addition, the self-powered triboelectric nanogenerator can be used in smart home and Internet of things technology fields to achieve continuous data monitoring, and can also be used in industrial automation equipment to reduce maintenance requirements and improve industrial automation efficiency. Since the triboelectric nanogenerator can be self-powered, its service life can be extended, costs can be reduced, and the inconvenience of replacing the power supply (battery) can be prevented.

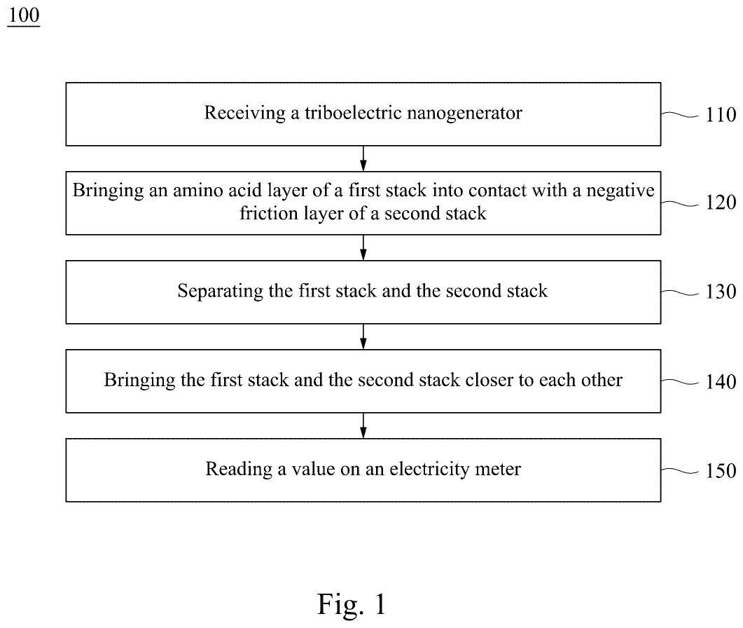

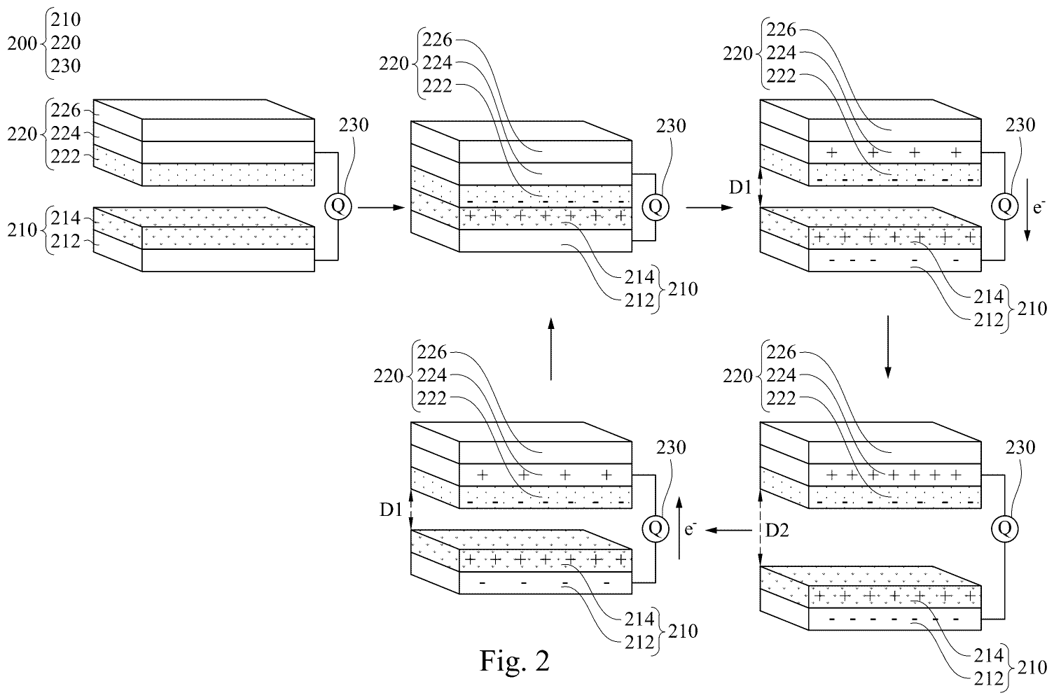

The present disclosure provides a triboelectric nanogenerator and an operation method thereof. Please refer to both FIG. 1 and FIG. 2. FIG. 1 is a flow diagram of a method 100 of operating a triboelectric nanogenerator according to various embodiments of the present disclosure. The method 100 includes operations 110, 120, 130, 140, and 150. FIG. 2 is a schematic diagram of various intermediate stages of operating a triboelectric nanogenerator 200 in accordance with various embodiments of the present disclosure.

In operation 110, as shown in FIG. 2, the triboelectric nanogenerator 200 is received. The triboelectric nanogenerator 200 includes a first stack 210, a second stack 220, and an electricity meter 230. The first stack 210 includes a first conductive layer 212 and an amino acid layer 214 disposed on the first conductive layer 212, in which the amino acid layer 214 is an object to be tested. The second stack 220 is disposed over the first stack 210 and is spaced apart from the first stack 210, in which the second stack 220 includes a negative friction layer 222, a second conductive layer 224 disposed on the negative friction layer 222, and an insulating layer 226 disposed on the second conductive layer 224. The electricity meter 230 is electrically connected to the first conductive layer 212 and the second conductive layer 224. In some embodiments, the electricity meter 230 is an electrometer.

Please continue to refer to FIG. 2. In some embodiments, the first conductive layer 212 includes a conductive oxide, such as indium tin oxide (ITO), indium zinc oxide (IZO), indium gallium oxide (IGO), indium gallium zinc oxide (IGZO), indium tin zinc oxide (ITZO), zinc tin oxide (ZTO), or combinations thereof, but is not limited thereto. In some embodiments, the first conductive layer 212 is a composite layer, such as glass attached with a conductive oxide. In some embodiments, the second conductive layer 224 includes, but not limited to, copper, silver, aluminum, gold, titanium, tungsten, or combinations thereof. The negative friction layer 222 is a friction layer that is negatively charged after contacting the amino acid layer 214. In some embodiments, the negative friction layer 222 includes polytetrafluoroethylene (PTFE), fluorinated ethylene propylene (FEP), polydimethylsiloxane (PDMS), silicone, poly(butylene adipate-co-terephthalate) (PBAT), polyvinylidene difluoride (PVDF), polyimide (PI), polystyrene (PS), polycarbonate (PC), or combinations thereof. In some embodiments, the negative friction layer 222 has a thickness of 10 nm to 1000 nm, such as 10, 50, 100, 200, 400, 600, 800, or 1000 nm. In some embodiments, the insulating layer 226 includes poly(methyl methacrylate) (PMMA), polyethylene terephthalate, polyethylene, polyethersulfone, polycarbonate, polyimide, or combination thereof. In some implementations, the insulating layer 226 is omitted.

In operation 120, as shown in FIG. 2, the amino acid layer 214 of the first stack 210 is brought into contact with the negative friction layer 222 of the second stack 220, in which the amino acid layer 214 and the negative friction layer 222 include different materials. Since the work function of the negative friction layer 222 is higher than the work function of the amino acid layer 214, this contact generates triboelectric charges. In FIG. 2, the charges shown in the negative friction layer 222 and the amino acid layer 214 are triboelectric charges. In more detail, after the amino acid layer 214 and the negative friction layer 222 are in contact with each other, electrons are transferred from the amino acid layer 214 to the negative friction layer 222, thereby making the negative friction layer 222 be negatively charged and the amino acid layer 214 be positively charged. Therefore, the amino acid layer 214 is a positive friction layer. In some embodiments, the amino acid layer 214 includes a L-type amino acid or a D-type amino acid. In some embodiments, the amino acid layer 214 includes glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline, or combinations thereof. In some embodiments, the amino acid layer 214 has a thickness of 1 nm to 1000 nm, such as 10, 50, 100, 200, 400, 600, 800, or 1000 nm. The triboelectric nanogenerator 200 of the present disclosure can analyze and identify different types of amino acids and can also analyze and identify amino acids with different chiralities; in other words, it can analyze and identify different stereoisomers of amino acids.

The stratum corneum of human skin may include glycine, L-alanine, L-histidine, L-threonine, L-proline, L-aspartic acid, and L-serine. In addition, as people age, D-aspartic acid in the stratum corneum may gradually increase, leading to skin aging. In some embodiments, human skin debris can be directly adhered to the first conductive layer 212 to form the amino acid layer 214. In some embodiments, human hair can be directly adhered to the first conductive layer 212 to form the amino acid layer 214. In some embodiments, human hair (e.g., head hair) can be dissolved and then coated on the first conductive layer 212 to form the amino acid layer 214. Since the triboelectric nanogenerator 200 of the present disclosure can analyze and identify amino acids, it can be applied in the field of health detection to analyze and detect the status of human skin and hair. The triboelectric nanogenerator 200 of the present disclosure can be used as an implantable device to be buried inside the human body for health detection. Since it does not need to be connected to an external power supply or equipped with a battery, it has the advantage of long service life and can prevent the harm to the human body caused by replacing a power supply (battery).

In operation 130, as shown in FIG. 2, the first stack 210 and the second stack 220 are separated. When the distance D1 between the first stack 210 and the second stack 220 gradually becomes larger, electrons flow from the second conductive layer 224 to the first conductive layer 212 in order to shield the triboelectric charge change. In FIG. 2, the charges shown in the first conductive layer 212 and the second conductive layer 224 are electrostatic charges. When the distance D1 between the first stack 210 and the second stack 220 increases to greater than or equal to the limit distance D2 (maximum separation distance), for example, ten times the thickness of the negative friction layer 222, almost all electrons generated by the triboelectric charging are transferred to the second conductive layer 224 and then flow into the first conductive layer 212. In operation 140, the first stack 210 and the second stack 220 are brought closer to each other, and electrons flow from the first conductive layer 212 into the second conductive layer 224. In operation 150, as shown in FIG. 2, a value (such as current value, voltage value, or both) on the electricity meter 230 is read to obtain the transfer charge and triboelectric charge density (TECD), thereby establishing triboelectric series of different amino acids. In more detail, during performing operations 120, 130, and 140, operation 150 is simultaneously performed to obtain the output current from the electricity meter 230. In some embodiments, operations 120 to 150 are performed repeatedly. Since different amino acids have different abilities to donate and accept electrons, the transfer charges are also different. Therefore, the triboelectric nanogenerator 200 can analyze and identify different types of amino acids, and it can also analyze and identify different stereoisomers of amino acids.

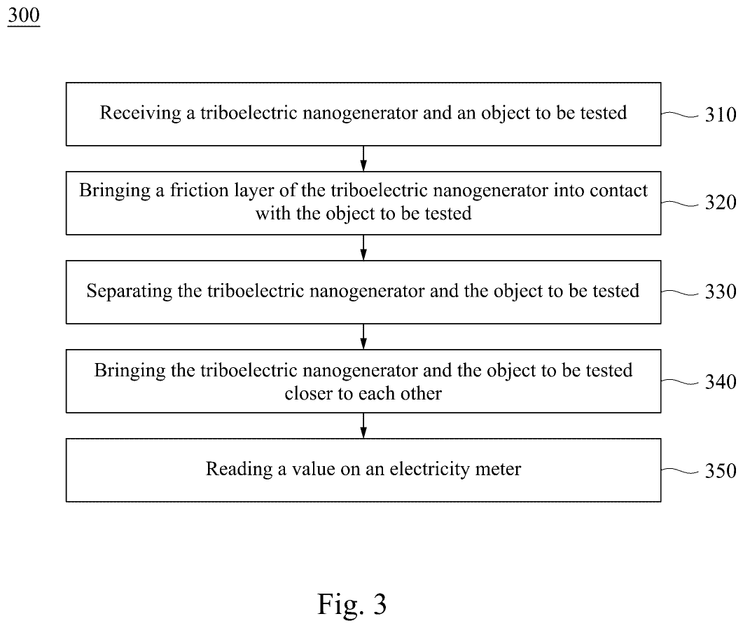

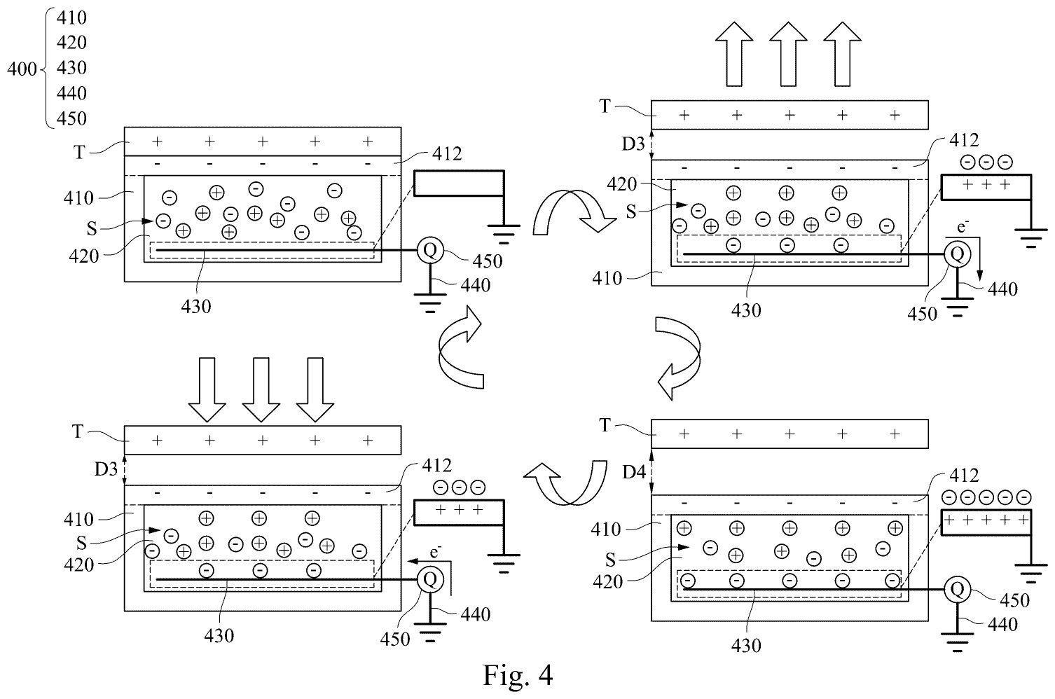

The present disclosure provides another triboelectric nanogenerator and a method of operating the same. Please refer to both FIG. 3 and FIG. 4. FIG. 3 is a flow diagram of a method 300 of operating a triboelectric nanogenerator according to various embodiments of the present disclosure. The method 300 includes operations 310, 320, 330, 340, and 350. FIG. 4 is a schematic diagram of various intermediate stages of operating a triboelectric nanogenerator 400 in accordance with various embodiments of the present disclosure. FIG. 4 shows schematic cross-sections of the triboelectric nanogenerator 400 during operation.

In operation 310, as shown in FIG. 4, the triboelectric nanogenerator 400 and an object to be tested T are received. The triboelectric nanogenerator 400 includes a packaging container 410, a filler 420, an electrode 430, a wire 440 and an electricity meter 450. The packaging container 410 has an accommodation space S and a friction layer 412. In other embodiments, a portion (e.g., the top portion) of the packaging container 410 is the friction layer 412 and the remaining portion includes a material different from the friction layer 412. In some embodiments, the friction layer 412 and the remaining portion of the packaging container 410 include the same material. In some embodiments, the upper surface of the friction layer 412 is a substantially flat surface as shown in FIG. 4. In other embodiments, the upper surface of the friction layer 412 has a plurality of protrusions protruding outwardly (not shown). The filler 420 is contained in the accommodation space S and is in contact with the friction layer 412, in which the filler 420 includes water, an electrolyte, or a liquid metal. The electrolyte may include a salt solution, an ionic liquid, an acidic solution, or an alkaline solution. The salt solution may be an aqueous salt solution, and may include, for example, the following salts: sodium chloride (NaCl), sodium sulfate (Na₂SO₄), potassium chloride (KCl), calcium chloride (CaCl₂)), potassium nitrate (KNO₃), magnesium sulfate (MgSO₄), sodium acetate (CH₃COONa), sodium carbonate (Na₂CO₃), sodium hydroxide (NaOH), sodium nitrate (NaNO₃), aluminium sulfate (Al₂(SO₄)₃), aluminum chloride (AlCl₃), potassium sulfate (K₂SO₄), potassium carbonate (K₂CO₃), sodium phosphate (Na₃PO₄), ammonium chloride (NH₄Cl), ammonium nitrate (NH₄NO₃), magnesium chloride (MgCl₂), copper sulfate (CuSO₄), iron (III) chloride (FeCl₃), potassium phosphate (K₃PO₄), or combinations thereof. The ionic liquid may include, for example, a sodium chloride ionic liquid or other salts with low volatility, high conductivity, good thermal stability, and a wide liquid range, such as 1-butyl-3-methylimidazolium hexafluorophosphate ([BMIM][PF₆]), 1-ethyl-3-methylimidazolium tetrafluoroborate ([EMIM][BF₄]), 1-butyl-3-methylimidazolium acetate ([BMIM][OAc]), 1-ethyl-3-methylimidazolium ethyl sulfate ([EMIM][EtSO₄]), 1-butyl-3-methylimidazolium chloride ([BMIM][Cl]), N-methylpyrrolidinium bis(trifluoromethylsulfonyl)imide ([PYR₁][TFSI]), 1-butyl-3-methylimidazolium thiocyanate ([BMIM][SCN]), tetraethylammonium (Et₄N⁺), 1-ethyl-3-methylimidazolium dicyanamide ([EMIM][dca]), tetramethylammonium ([N₁₁₁₁][TFSI]), 1-ethyl-3-methylimidazolium bis(trifluoromethylsulfonyl)imide ([EMIM][NTf₂]), 1-butylpyridinium hexafluorophosphate ([BPY][PF₆]), 1-hexyl-3-methylimidazolium trifluoroacetate ([HMIM][CF₃COO]), 1-propyl-3-methyl-imidazolium bis(trifluoromethylsulfonyl)imide ([PMIM][NTf₂]), 1-methylpyrrolidinium tetrafluoroborate ([PYR₁][BF₄]), 1-methylpyrrolidinium trifluoromethanesulfonate ([PYR₁][CF₃SO₃]), tetrabutylammonium bis(trifluoromethylsulfonyl)imide ([TBA][NTf₂]), 1-hexyl-3-methylimidazolium acetate ([HMIM][OAc]), 1-butyl-1-methylpiperidinium hexafluorophosphate ([BMP][PF₆]), 1-propyl-3-methylimidazolium chloride ([PMIM][Cl]), or combinations thereof. The liquid metal may include Ga, In, or Sn, for example. In some embodiments, the filler 420 fills the accommodation space S. The filler 420 serves as an electron conduction medium and has high electron conductivity, thereby improving the measurement sensitivity of the triboelectric nanogenerator 400. The electrode 430 penetrates the packaging container 410 and is in contact with the filler 420. The wire 440 is electrically connected to the electrode 430 and has a grounded end. The electricity meter 450 is disposed on the wire 440 to measure the current flowing through the wire 440.

Please continue to refer to FIG. 4. In some embodiments, the packaging container 410 and the friction layer 412 each include polytetrafluoroethylene (PTFE), fluorinated ethylene propylene (FEP), polydimethylsiloxane (PDMS), silicone, poly(butylene adipate-co-terephthalate) (PBAT), polyvinylidene difluoride (PVDF), polyimide (PI), polystyrene (PS), polycarbonate (PC), or combinations thereof, so it can have good biocompatibility. For example, the friction layer 412 includes silicone Ecoflex. The triboelectric nanogenerator 400 can be installed on, for example, a robot, a mechanical finger, or a wearable device. In some embodiments, the packaging container 410 and the friction layer 412 are stretchable and flexible, so when the robot, the mechanical finger, or a part wearing the wearable device moves, the triboelectric nanogenerator 400 can still perform sensing and thus have good applicability. The electrode 430 and the wire 440 independently include copper, silver, aluminum, gold, titanium, tungsten or combinations thereof, but are not limited thereto.

Please continue to refer to FIG. 4. In some embodiments, the filler 420 is a salt solution, and the salt solution includes a salt in a concentration of 0.05 wt % to 10 wt %, such as 0.05, 0.1, 0.2, 0.3, 0.4, 0.5, 0.6, 0.7, 0.8, 0.9, 1, 2, 4, 6, 8, or 10 wt %. When the salt concentration is within the above range, the salt solution can have high electronic conductivity. When the salt concentration is higher, the electronic conductivity of the salt solution is also higher.

In operation 320, as shown in FIG. 4, the friction layer 412 of the triboelectric nanogenerator 400 is brought into contact with the object to be tested T, in which the friction layer 412 and the object to be tested T have different materials. FIG. 4 shows a schematic diagram showing the generation of triboelectric charges because the work function of the friction layer 412 of the packaging container 410 is higher than the work function of the object to be tested T. In more detail, after the friction layer 412 and the object to be tested T are in contact with each other, electrons may transfer from the object to be tested T to the friction layer 412, so that the friction layer 412 is negatively charged, and the object to be tested T is positively charged. Therefore, the friction layer 412 is a negative friction layer, and the object to be tested T is a positive friction layer. In other embodiments, when the work function of the friction layer 412 of the packaging container 410 is lower than the work function of the object to be tested T, after the friction layer 412 and the object to be tested T are in contact with each other, electrons may transfer from the friction layer 412 to the object to be tested T, so that the friction layer 412 is positively charged, and the object to be tested T is negatively charged. Therefore, the friction layer 412 is a positive friction layer, and the object to be tested T is a negative friction layer (not shown). In some embodiments, the object to be tested includes a metal, a biomaterial, or a polymer. For example, the object to be tested T includes aluminum, copper, cotton, polysiloxane (polydimethylsiloxane, PDMS), polytetrafluoroethylene (PTFE), fluorinated ethylene propylene (FEP), an amino acid, a peptide, a protein, skin, hair, head hair, or combinations thereof, but is not limited thereto.

In operation 330, as shown in FIG. 4, the triboelectric nanogenerator 400 and the object to be tested T are separated. During separation, in the filler 420, cations accumulate near the interface between the filler 420 and the friction layer 412, and anions accumulate near the interface between the filler 420 and the electrode 430. Electrons flow to the ground via the electrode 430 to establish charge balance. When the distance D3 between the triboelectric nanogenerator 400 and the object to be tested T increases to greater than or equal to the limit distance D4, for example, ten times the thickness of the friction layer 412, charge balance is reached, and the electrons stop flowing to the ground. In operation 340, as shown in FIG. 4, the triboelectric nanogenerator 400 and the object to be tested T are brought closer to each other. The amount of the induced charges on the electrode 430 decreases, causing electrons to flow back from the ground to the electrode 430. In operation 350, as shown in FIG. 4, a value (e.g., current value, voltage value, or both) on the electricity meter 450 is read to obtain the transfer charge. In more detail, during performing operations 320, 330, and 340, operation 350 is simultaneously performed to obtain the output current from the electricity meter 450. In some embodiments, operations 320 to 350 are repeated. Since different objects to be tested have different abilities to donate and accept electrons, the transfer charges are also different. Therefore, the triboelectric nanogenerator 400 can analyze and identify objects to be tested of different materials.

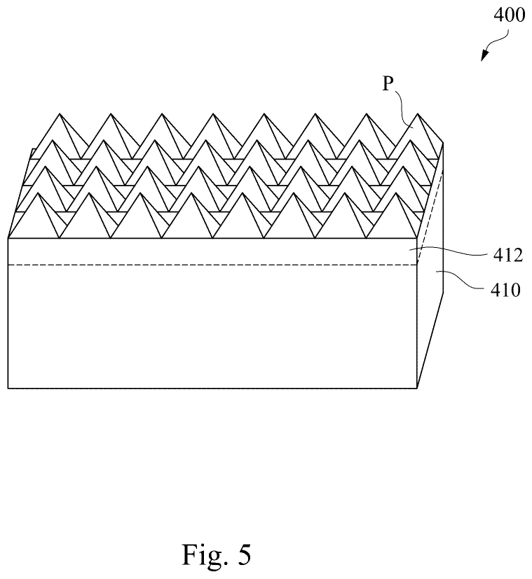

FIG. 5 is a perspective view of the packaging container 410 of the triboelectric nanogenerator 400 according to various embodiments of the present disclosure. In some embodiments, the friction layer 412 has a plurality of protrusions P (which may also be referred to as a protrusion array) protruding outwardly. The number of the protrusions P is not limited to the number shown in FIG. 5. The number of the protrusions P can be adjusted arbitrarily according to design requirements. The protrusions P can increase the contact area between the friction layer 412 and the object to be tested T and increase the hydrophobicity of the friction layer 412, thereby improving the measurement sensitivity of the triboelectric nanogenerator 400. In some embodiments, the protrusions P are microcones. In some embodiments, the protrusions P are pyramid-shaped, conical, or cylindrical, but are not limited thereto.

The following describes the features of the present disclosure more specifically with reference to Experimental Examples 1 to 4. Although the following examples are described, the materials, their amounts and ratios, processing details, processing procedures, etc., may be appropriately varied without exceeding the scope of the present disclosure. Accordingly, the present disclosure should not be interpreted restrictively by the experimental examples described below.

### Experimental Example 1: Measurement of Transfer Charges, Transfer Charge Densities, Surface Potentials, and Work Functions of Amino Acids

Different amino acids were measured with the triboelectric nanogenerator 200 shown in FIG. 2. In the triboelectric nanogenerator 200 of Experimental Example 1, glass attached with an ITO layer (ITO glass) was used as the first conductive layer 212, a PTFE layer was used as the negative friction layer 222, a copper layer was used as the second conductive layer 224, a PMMA layer was used as the insulating layer 226, and an electrometer (Keithley Model 6514) was used as the electricity meter 230. The PTFE layer has a work function of 5.8 eV. The manufacturing method of the triboelectric nanogenerator 200 included the following steps. First, the ITO glass (1 cm×1.5 cm×0.04 cm) was soaked in deionized water, acetone, and ethanol for 10 minutes each for ultrasonic cleaning. The ITO glass was then spray-dried using nitrogen gas. The conductive surface of the ITO glass was treated with oxygen plasma at a power of 28 W and an O₂ pressure of 6.7×10⁻¹ bar for 5 minutes to improve the hydrophilicity of the conductive surface. Next, a 25 mM amino acid aqueous solution was coated on the ITO surface by spin coating, in which the aqueous solution was spun at 200 rpm for 30 seconds and then spun at 1000 rpm for 10 seconds, to form the amino acid layer 214 with a thickness of about 400 nm, thereby forming the first stack 210. The first stack 210 was dried at 37° C. for 5 hours. In addition, the method of manufacturing the second stack 220 included the following steps. The copper layer was attached to the PMMA layer, and then the PTFE layer (1 cm×1.5 cm×0.013 cm) was adhered to the copper layer. The electricity meter 230 was electrically connected to the copper layer and ITO layer by a copper wire.

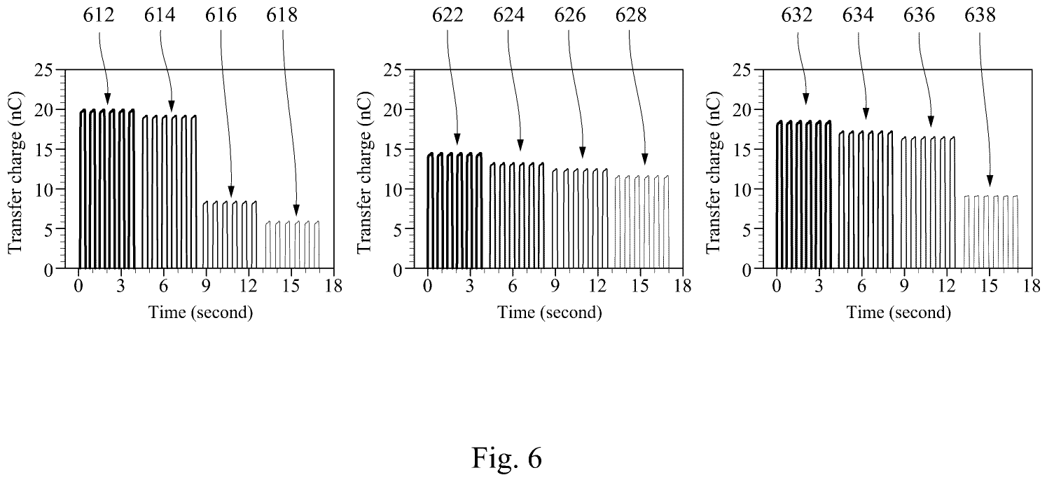

Please refer to FIG. 1 to operate the triboelectric nanogenerator 200. Operations 120 to 150 are a cycle. Six cycles were repeated, and the transfer charges of different amino acids were recorded, in which a linear motor controlled by a program performed the contact and separation of the first stack 210 and the second stack 220 and was operated with a force of 1 N and a frequency of 2 Hz. The separation speed was 0.0256 meters/second, the maximum separation distance was 1 cm, and the contact area was (1×1.5) cm². FIG. 6 shows transfer charge versus time plots of various amino acids. The curves 612, 614, 616, and 618 respectively show the measurement results of L-arginine, L-histidine, L-glutamic acid, and L-aspartic acid. These amino acids are amino acids having charged side chain groups. The curves 622, 624, 626, and 628 respectively show the measurement results of L-serine, L-glutamine, L-threonine, and L-asparagine. These amino acids are amino acids having uncharged and polar side chain groups. The curves 632, 634, 636, and 638 respectively show the measurement results of L-proline, L-alanine, glycine, and L-cysteine. These amino acids are amino acids having hydrophobic side chain groups. It can be seen that the transfer charges of different amino acids are significantly different, so different amino acids can be identified by the triboelectric nanogenerator 200.

The surface potentials of the above 12 amino acids were measured using Kelvin probe force microscopy (KPFM). The work functions of the above 12 amino acids were measured by measure the contact potential difference (CPD) between the cantilever probe of the atomic force microscope (AFM) (single crystal diamond conductive probe, AD-2.8-AS probe) and the amino acid samples. CPD=(the work function of the cantilever probe−the work function of the amino acid sample)/the basic charge of the electron. Please refer to Table 1 below for the surface potential and the work function of each amino acid. It can be seen from Table 1 that the surface potentials and work functions are inversely proportional.

TABLE 1

| | Surface | Work |
|---|---|---|
| | potential | function |
| | (mV) | (eV) |
| L-Arginine | 1214 | 3.786 |
| L-Histidine | 1177 | 3.823 |
| L-Proline | 1056 | 3.944 |
| L-Alanine | 909 | 4.091 |
| Glycine | 761 | 4.239 |
| L-Serine | 701 | 4.299 |
| L-Glutamine | 571 | 4.429 |
| L-Threonine | 373 | 4.627 |
| L-Asparagine | 271 | 4.729 |
| L-Cysteine | 199 | 4.801 |
| L-Glutamic acid | 101 | 4.899 |
| L-Aspartic acid | 26 | 4.974 |

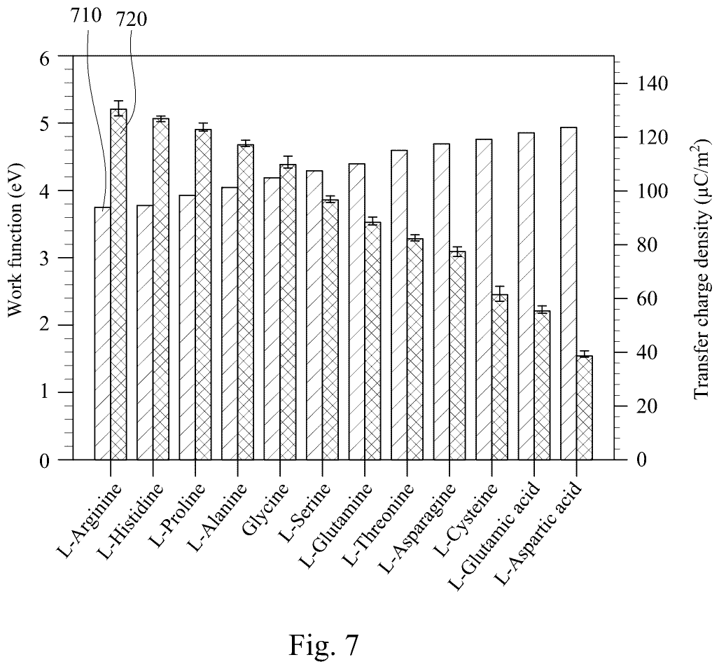

According to FIG. 6, the triboelectric charge densities (TECDs) of different amino acids can be further calculated. Next, please refer to FIG. 7, which shows the work functions and transfer charge densities of various amino acids. The bars 710 correspond to the work functions of the amino acids, and the bars 720 correspond to the transfer charge densities of the amino acids. It can be seen that the work functions of the amino acids are inversely proportional to the transfer charge densities of the amino acids. When an amino acid has a higher work function, it has a lower transfer charge density. In other words, when the work function of the amino acid is closer to the work function of the negative friction layer 222, the transfer charge density is lower. In FIG. 7, the amino acid closer to the left has a stronger electron donating ability.

### Experimental Example 2: Measurement of Transfer Charges, Transfer Charge Densities, and Output Voltages of D-Aspartic Acid and L-Aspartic Acid

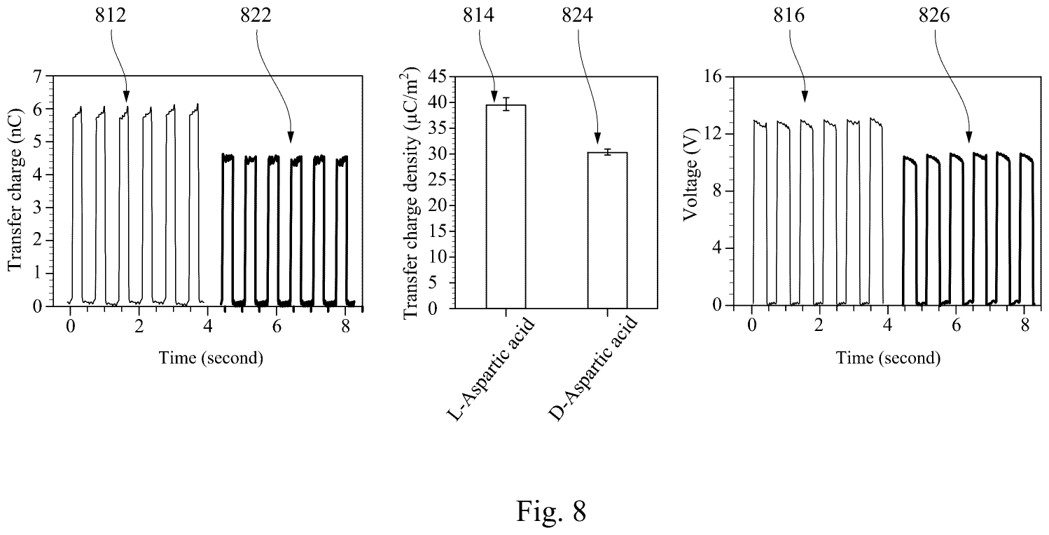

D-aspartic acid and L-aspartic acid were measured using the triboelectric nanogenerator 200 and the measurement method of Experimental Example 1. FIG. 8 shows a transfer charge versus time relationship diagram, transfer charge densities, and an output voltage versus time relationship diagram of the D-aspartic acid and L-aspartic acid. The work function of D-aspartic acid is higher than that of L-aspartic acid. The curves 812, 814, and 816 show the measurement results of L-aspartic acid, and the curves 822, 824, and 826 show the measurement results of D-aspartic acid. It can be seen from FIG. 8 that the transfer charge and transfer charge density of D-aspartic acid are smaller than the transfer charge and transfer charge density of L-aspartic acid. Furthermore, the output voltage of D-aspartic acid in the open circuit state is smaller than the output voltage of L-aspartic acid. It can be seen that different stereoisomers of amino acids can be easily distinguished by the triboelectric nanogenerator 200. The triboelectric nanogenerator 200 can be used in the fields of biomedicine and environmental science.

### Experimental Example 3: Measurement of Aluminum Layer with Triboelectric Nanogenerator Under Different Experimental Conditions

The object to be tested T was measured with the triboelectric nanogenerator 400 shown in FIG. 4. In the triboelectric nanogenerator 400 of Experimental Example 3, silicone Ecoflex 00-30 was used as the material of the packaging container 410, a NaCl aqueous solution was used as the filler 420 (conductive medium), a copper electrode was used as the electrode 430, and an aluminum layer was used as the object to be tested T. The manufacturing method of triboelectric nanogenerator 400 (3 cm×3 cm×2 mm) included the following steps. First, a Ecoflex prepolymer and a curing agent were mixed at a volume ratio of 1:1, then filled into two different PDMS molds, and cured at 80° C. for 1 hour. One PDMS mold was used to manufacture the lower half of the packaging container 410, and the other PDMS mold was used to manufacture the upper half of the packaging container 410, that is, the upper half of the packaging container 410 with the friction layer 412, in which the friction layer 412 has a plurality of pyramid-shaped protrusions. The schematic diagram of the pyramid-shaped protrusions is shown in FIG. 5. The lower half and the upper half were taken out from the molds, bonded together with uncured Ecoflex, and cure at 80° C. for 1 hour to obtain the packaging container 410. A NaCl aqueous solution was injected into the accommodation space S of the packaging container 410. The electrode 430, the wire 440, and the electricity meter 450 are further provided.

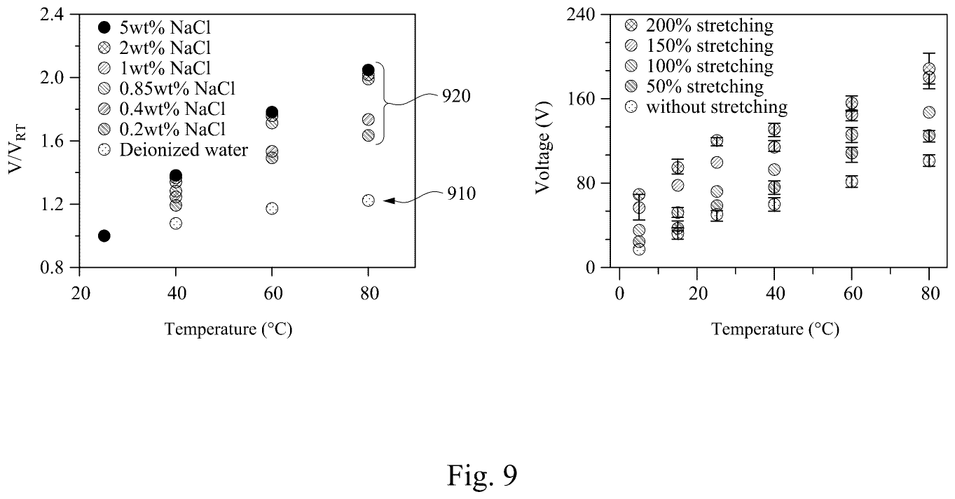

Please refer to FIG. 3 to operate the triboelectric nanogenerator 400. Operations 320 to 350 are a cycle. Cycles were repeated, and the voltages and currents were recorded. A linear motor was used to perform contact and separation between the triboelectric nanogenerator 400 and the object to be tested T. FIG. 9 shows a V/V_{RT} versus temperature relationship diagram when NaCl aqueous solutions of different concentrations and deionized water were used as the filler 420 of the triboelectric nanogenerator 400. As shown in FIG. 9, the deionized water was used as the filler 420 to measure the object to be tested T (aluminum layer) at different temperatures. For the measurement results, please refer to the data points 910. The NaCl aqueous solutions with different weight percentage concentrations were used as the filler 420 to measure the object to be tested T (aluminum layer) at different temperatures. For the measurement results, please refer to the data points 920. The friction layer 412 serves as a negative friction layer, and the aluminum layer serves as a positive friction layer. It can be seen from FIG. 9 that when the NaCl concentration in the NaCl aqueous solution is higher, the output voltage/output voltage at normal temperature (23° C.) (V/V_{RT}) is higher. It can be seen that the increase in NaCl concentration can improve the sensitivity of the triboelectric nanogenerator 400. In addition, as can be seen from FIG. 9, the triboelectric nanogenerator 400 can identify aluminum layers at different temperatures through different signal intensities. FIG. 9 shows the output voltage versus temperature relationship diagram of the triboelectric nanogenerator 400 under different stretching degrees, in which 1 wt % NaCl aqueous solution was used as the filler 420. The silicone Ecoflex 00-30 is stretchable. It can be seen from FIG. 9 that under different stretching rates, the triboelectric nanogenerator 400 can still identify the aluminum layers at different temperatures through different signal intensities.

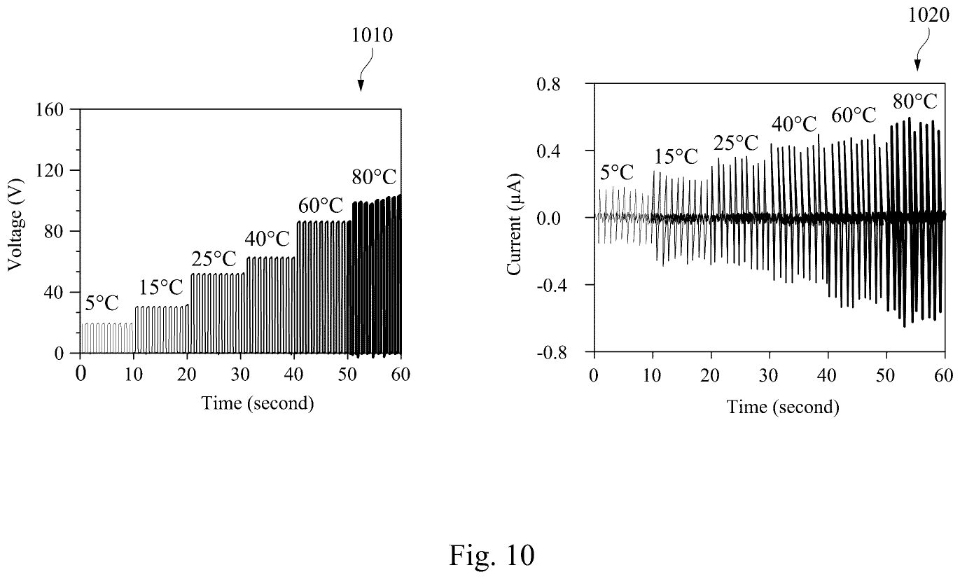

FIG. 10 shows an open circuit voltage versus time relationship diagram and a short-circuit current versus time relationship diagram of the triboelectric nanogenerator 400 at different temperatures, in which a 1 wt % NaCl aqueous solution was used as the filler 420. Please refer to the curve 1010 for the measurement results of the open circuit voltage at different temperatures. Please refer to the curve 1020 for the measurement results of the short circuit current at different temperatures. It can be seen from FIG. 10 that when the temperature of the aluminum layer is higher, the open circuit voltage and short-circuit current are higher. The triboelectric nanogenerator 400 can identify the aluminum layers at different temperatures through different open circuit voltage intensities or different short-circuit current intensities. The sensing capability of the triboelectric nanogenerator 400 increases as the temperature increases.

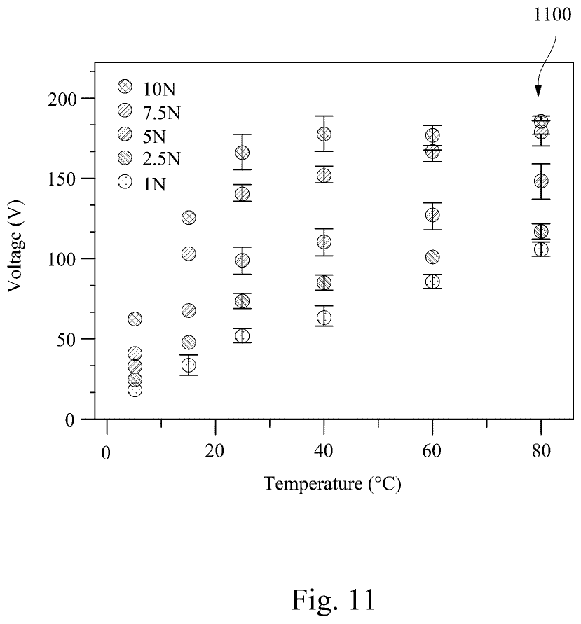

FIG. 11 shows an output voltage versus temperature relationship diagram of the triboelectric nanogenerator 400 under different forces, in which a 1 wt % NaCl aqueous solution was used as the filler 420. Please refer to the data points 1100 for the measurement results of the open circuit voltage under different forces. Silicone Ecoflex 00-30 is stretchable. It can be seen from FIG. 11 that when the silicone Ecoflex 00-30 was subjected to different forces, the triboelectric nanogenerator 400 can still identify the aluminum layers of different temperatures through different signal intensities and has good sensitivity. In addition, after the triboelectric nanogenerator 400 is folded, twisted, or bent, the value of the open circuit voltage remains roughly unchanged after 500 cycles (operations 320 to 350 are one cycle). During a 7-day continuous test of triboelectric nanogenerator 400, the open circuit voltage value remained roughly unchanged. The above measurement results represent that the triboelectric nanogenerator 400 has good stability.

### Experimental Example 4: Measurement of Different Objects to be Tested Under Different Experimental Conditions by Using Triboelectric Nanogenerator

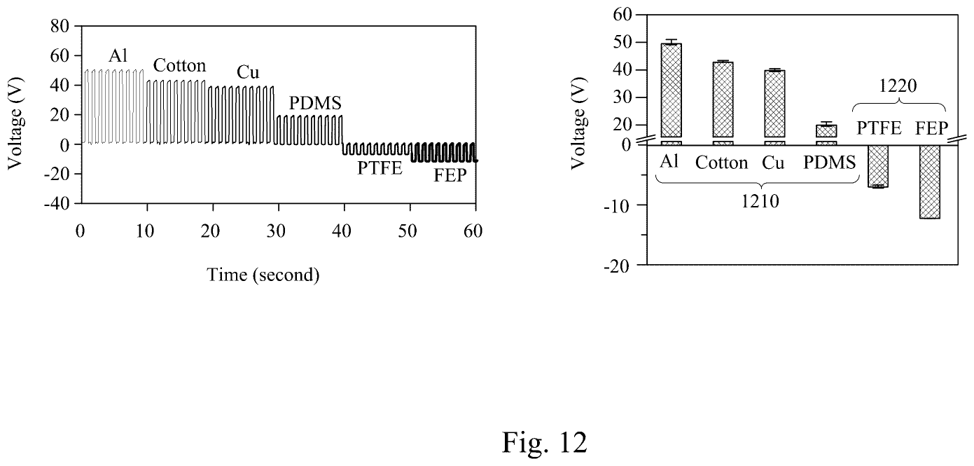

Different objects to be tested were measured using the triboelectric nanogenerator 400 and the measurement method of Experimental Example 3. FIG. 12 shows an output voltage versus time relationship diagram and an average open circuit voltage diagram of the triboelectric nanogenerator 400 when measuring different objects to be tested. As shown in FIG. 12, the open circuit voltages of aluminum (AI), cotton, copper (Cu), polysiloxane (PDMS), polytetrafluoroethylene (PTFE), and fluorinated ethylene propylene (FEP) are different. When measuring Al, cotton, Cu, and PDMS, these objects to be tested belong to the positive friction layers 1210. When measuring PTFE and FEP, these objects to be tested belong to the negative friction layers 1220. The output voltages of Al, cotton, Cu, and PDMS are about 50 V, 42 V, 39 V, and 19 V, respectively, and the output voltages of PTFE and FEP are about −7 V and −12 V, respectively. Therefore, the triboelectric nanogenerator 400 can be used to identify different objects to be tested.

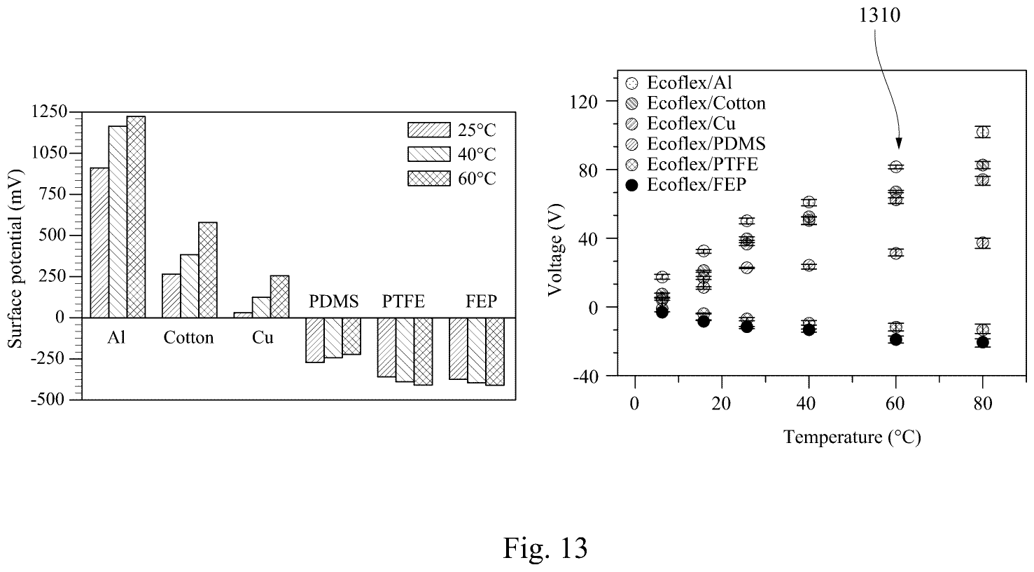

FIG. 13 shows a surface potential diagram of various objects to be tested at different temperatures and an output voltage versus temperature relationship diagram of different objects to be tested. It can be seen from FIG. 13 that different objects to be tested have different surface potentials and different open circuit voltages. A single object to be tested has different surface potentials and different open circuit voltages at different temperatures (25° C., 40° C., 60° C.). The surface potentials of Al, cotton, Cu, and PDMS increase as the temperature rises, while the surface potentials of PTFE and FEP decrease as the temperature rises. Please refer to the data points 1310 for the measurement results of the open circuit voltage. Therefore, the triboelectric nanogenerator 400 can be used to identify different objects to be tested.

The surface potentials of the above objects to be tested were measured with Kelvin probe force microscopy (KPFM). The work functions of the objects to be tested were measured by measuring the contact potential difference (CPD) between the cantilever probe of the atomic force microscope (AFM) (single crystal diamond conductive probe, AD-2.8-AS probe) and the samples of the objects to be tested. CPD=(the work function of the cantilever probe−the work function of the sample)/the basic charge of the electron. Please refer to Table 2 below for the surface potentials and work functions of different objects to be tested at room temperature of 25 degrees. It can be seen from Table 2 that the surface potentials and work functions are inversely proportional.

TABLE 2

| | Surface | Work |
|---|---|---|
| | potential | function |
| | (mV) | (eV) |
| Al | 918 | 4.082 |
| Cotton | 275 | 4.725 |
| Cu | 39 | 4.961 |
| Polydimethylsiloxane (PDMS) | −273 | 5.273 |
| Ecoflex | −304 | 5.304 |
| Polytetrafluoroethylene (PTFE) | −364 | 5.364 |
| Fluorinated ethylene propylene (FEP) | −376 | 5.376 |

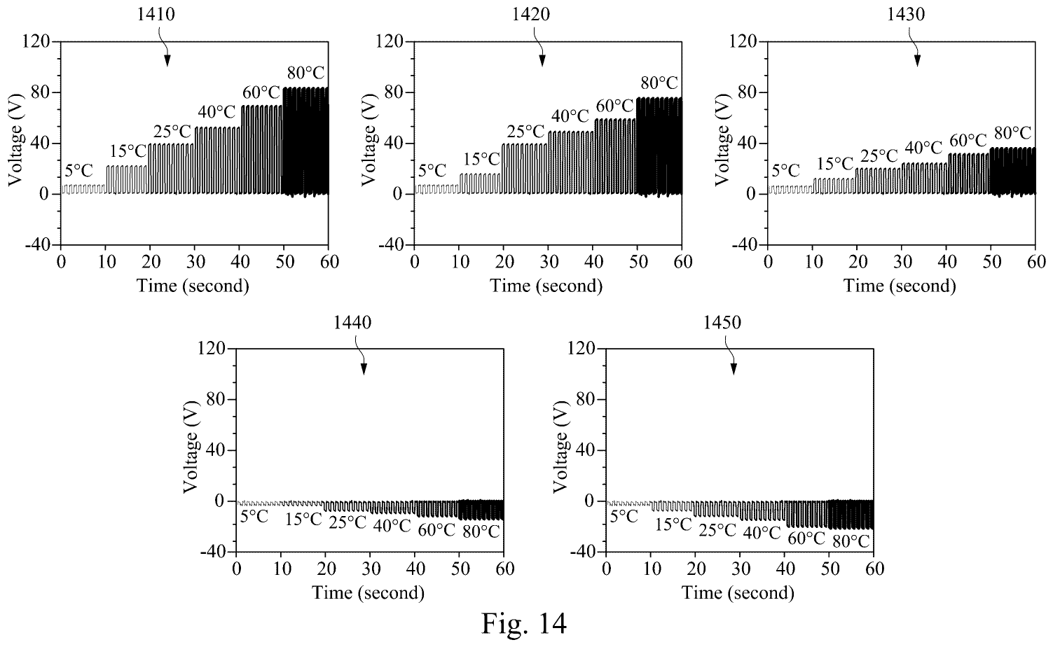

FIG. 14 shows output voltage versus time relationship diagrams of the triboelectric nanogenerator 400 when measuring various objects to be tested. For the measurement results of cotton, Cu, PDMS, PTFE, and FEP, please refer to the curves 1410, 1420, 1430, 1440, and 1450 respectively. Therefore, the triboelectric nanogenerator 400 can be used to identify different objects to be tested and identify temperature differences.

In summary, the present disclosure provides a triboelectric nanogenerator and an operation method thereof. The triboelectric nanogenerator can identify different objects to be tested and different temperatures through measured data (such as voltage, current, transfer charge) and has the advantages of high sensitivity, high stability, low cost, and self-power supply, so it can be applied in, for example, health monitoring fields, environmental monitoring fields, and industrial automation equipment.

Although the present disclosure has been described in considerable detail with reference to certain embodiments, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present disclosure cover the modifications and variations of the present disclosure falling within the scope of the appended claims.

What is claimed is:

1. A triboelectric nanogenerator, comprising:
a first stack comprising:
a first conductive layer; and
an amino acid layer disposed on the first conductive layer;
a second stack disposed over the first stack, wherein the second stack comprises:
a negative friction layer; and
a second conductive layer disposed on the negative friction layer; and
an electricity meter electrically connected to the first conductive layer and the second conductive layer.

2. The triboelectric nanogenerator of claim 1, wherein the negative friction layer comprises polytetrafluoroethylene, fluorinated ethylene propylene, polydimethylsiloxane, silicone, poly(butylene adipate-co-terephthalate), polyvinylidene difluoride, polyimide, polystyrene, polycarbonate, or combinations thereof.

3. The triboelectric nanogenerator of claim 1, wherein the amino acid layer comprises a L-type amino acid or a D-type amino acid.

4. The triboelectric nanogenerator of claim 1, wherein the amino acid layer comprises glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline, or combinations thereof.

5. The triboelectric nanogenerator of claim 1, wherein the negative friction layer has a thickness of 10 nm to 1000 nm.

6. The triboelectric nanogenerator of claim 1, wherein the amino acid layer has a thickness of 1 nm to 1000 nm.

7. The triboelectric nanogenerator of claim 1, wherein a work function of the negative friction layer is higher than a work function of the amino acid layer.

8. A method of operating a triboelectric nanogenerator, comprising:
receiving the triboelectric nanogenerator of claim 1;
bringing the amino acid layer of the first stack into contact with the negative friction layer of the second stack;
separating the first stack and the second stack;
bringing the first stack and the second stack closer to each other; and
reading a value on the electricity meter.

9. The method of claim 8, wherein the value is a current value, a voltage value, or both.

10. A triboelectric nanogenerator, comprising:
a packaging container having an accommodation space and a friction layer;
a filler contained in the accommodation space and in contact with the friction layer, wherein the filler comprises water, an electrolyte, or a liquid metal;
an electrode penetrating the packaging container and being in contact with the filler;
a wire electrically connected to the electrode and having a grounded end; and
an electricity meter disposed on the wire.

11. The triboelectric nanogenerator of claim 10, wherein the friction layer comprises polytetrafluoroethylene, fluorinated ethylene propylene, polydimethylsiloxane, silicone, poly(butylene adipate-co-terephthalate), polyvinylidene difluoride, polyimide, polystyrene, polycarbonate, or combinations thereof.

12. The triboelectric nanogenerator of claim 10, wherein the friction layer has a plurality of protrusions protruding outwardly.

13. The triboelectric nanogenerator of claim 10, wherein the filler is a salt solution, and the salt solution comprises a salt in a concentration of 0.05 wt % to 10 wt %.

14. The triboelectric nanogenerator of claim 10, wherein the filler comprises water, an electrolyte, or a liquid metal.

15. The triboelectric nanogenerator of claim 14, wherein the electrolyte comprises a salt solution, an ionic liquid, an acidic solution, or an alkaline solution.

16. The triboelectric nanogenerator of claim 10, wherein the friction layer is a top portion of the packaging container and has a substantially flat upper surface.

17. A method of operating a triboelectric nanogenerator, comprising:
receiving the triboelectric nanogenerator of claim 10 and an object to be tested;
bringing the friction layer of the triboelectric nanogenerator into contact with the object to be tested;
separating the triboelectric nanogenerator and the object to be tested;
bringing the triboelectric nanogenerator and the object to be tested closer to each other; and
reading a value on the electricity meter.

18. The method of claim 17, wherein the value is a current value, a voltage value, or both.
